(19) United States
(12) Patent Application Publication
(10) Pub. No.: US 2022/0040360 A1
(43) Pub. Date: Feb. 10, 2022

(54) PORTABLE AIR STERILIZER WITH OXYGEN GENERATOR

(71) Applicant: Vandette B. CARTER, Yorktown Heights, NY (US)

(72) Inventor: Vandette B. CARTER, Yorktown Heights, NY (US)

(21) Appl. No.: 17/235,882

(22) Filed: Apr. 20, 2021

Related U.S. Application Data
(60) Provisional application No. 63/012,906, filed on Apr. 20, 2020.

(51) Int. Cl. A61L 9/20; A62B 7/10; A62B 9/00

(52) U.S. Cl. CPC A61L 9/20; A62B 7/10; A61L 2209/14; A61L 2209/15; A61L 2209/12; A62B 9/00

(57) ABSTRACT

A personal protective wearable air sterilizing apparatus is comprised of a series of looped internal conduits, designed in such a way to allow total internal reflection of UV light, and the simultaneous sterilization of atmospheric air. The apparatus has oxygen generator using electrolysis of water and neutralizes hydrogen gas derived from the process to an inert compound such as hydrogen peroxide; or reuses the hydrogen to generate electricity.

This application claims priority from and the benefit of provisional patent application Ser. No. 63/012,906 filed on Apr. 20, 2020, the disclosure of which is incorporated herein in its entirety, for all purposes.

### FIELD OF THE DISCLOSURE

The invention is in the field of PAPR (Personal Air Purifying Respirator) and (PPE) Personal Protective Equipment which are used to filter air to remove micro-pathogens, and particularly viruses.

### BACKGROUND

PPE and PAPR have become widely familiar abbreviations in the global vernacular due to the pandemic caused by the novel Covid-19 virus. PPE also include non-respirator type garments.

Since the emergence of Covid-19 (aka The Corona Virus) at the end of 2019, countries the world over have found themselves generally unprepared in various ways to combat this disease. The pandemic continues to ravage the globe and claimed more than 3,000,000 lives worldwide as of April, 2021. Though many countries' military can be described as ‘battle ready’, the medical community and other frontline workers were nowhere ready or equipped with the most basic of PPE (N95 mask) at the onset of the pandemic. There are numerous deeply personal and tragic stories covered by mainstream media and social media on the ill preparedness of many developed nations and the ongoing disparity for PPE or vaccines in poor countries. Frontline workers, have seen firsthand the numerous deaths and despair Covid-19 has left in its wake. Thanks to the effort of other medical professionals, several vaccines have become the frontline strategy to combat Covid-19 from reeking further havoc. Vaccines as we know work by training the body's immunity to fend off a disease, in this case, Covid-19. At least two vaccine candidates have proven efficacy greater than 90% while others have been marred with reports of complications or unwanted side effects. Since vaccines are not 100% protective, there is a need for frontline workers to be 100% protected, particularly when putting their own lives at risk to care for and treat those affected by a virulent microorganism such as Covid-19.

At the onset of the pandemic, health professionals globally, and more so in the USA found themselves in short supply of N95 masks, which in itself is not 100% protective from respiratory pathogens. The “95” designation denotes that the “best” face mask is certified to be 95% protective. In the worst case scenario, 5% of frontline worker will become infected with Covid-19 and approximately 5% (in line with national statistics in the United States) will lose their lives by wearing these masks while treating infected patients. Clearly these deductions do not sit well with any frontline worker, and the sad reality is that many frontline workers have lost their lives to Covid-19, with the majority of casualties occurring within the first 100 days of Covid-19 arrival on the shores of many countries, as a result of substandard PPE or lack thereof.

Besides not being 100% protective, N95 masks are “suffocating” to wear for any length of time. Most healthcare providers would have to wear one for 8-12 hours every day. The breathing issue becomes more complicated for providers who have underlying respiratory or cardiac conditions, or those who may be pregnant.

It is a known fact that variable levels of CO₂ (carbon dioxide) are retained in the mask and oxygen level percentage is decreases over time, making the task of performing essential and at times life-saving duties greatly challenging. N95 masks are by no means ideal to be used for the aforementioned reasons and based on the fact that there is a 5% or more chance that they will not prevent a virus such as Covid-19 from invading the respiratory system.

PAPR are more sophisticated battery-operated personal air purifying respirators (devices) which can cost upwards of $2,000 each. Unlike the case of military personnel, whose combat gear far exceeds this cost per person (sometimes by more than a factor of ten), PAPR are not readily available to front line workers, and their use may not be feasible, as was evident in nearly all private and public hospitals in the United States. One would argue that since threats like Ebola did reach the United States within the last decade, and H1N1 emerged within the last two decades, the arrival of Covid-19 or a similar pathogen seemed inevitable, as predicted by a handful of forward thinking people such as, for example, the Nobel Laurate Joshua Lederberg who said, “The single biggest threat to man . . . is the virus.” As such, nations should be preparing adequately to protect their frontline workers as a priority, for the inevitable confrontation with all viruses; those known and those yet to be known.

### SUMMARY OF THE DISCLOSURE

Disclosed herein is a personal protective apparatus, comprised of a series of looped internal conduits enclosed in a container, designed in such a way as to allow the passage and storage of large volume atmospheric air and oxygen, while permitting the transmission of UV light rays continuously within the labyrinth to sterilize the air, using the principle of total internal reflection (TIR). The apparatus can generate oxygen by electrolysis of water and has a system to neutralize the hydrogen gas derived from the process to create an inert substance such as hydrogen peroxide. The hydrogen can also be used to generate electricity in a fuel cell, which may be used to recharge a battery that powers the apparatus.

The disclosure is also directed to a portable apparatus which is designed to filter ambient air of virus particles using a novel approach.

### BRIEF DESCRIPTION OF THE FIGURES

FIG. 1 is a cross-sectional diagram of an apparatus described herein.

FIG. 2 illustrates the connection of the apparatus of FIG. 1 to a head covering face mask.

FIG. 3 illustrates the apparatus of FIG. 1 connected to a shoulder harness.

FIG. 4 is an illustration in which the apparatus of FIG. 1 connected to a shoulder harness of FIG. 3, can be worn in use.

### DETAILED DESCRIPTION OF THE PREFERRED EMBODIMENT

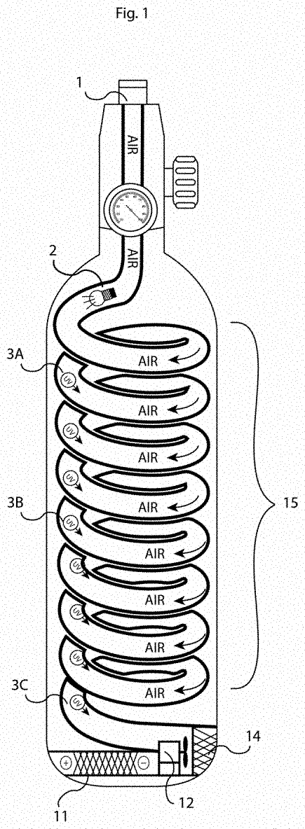

With reference to FIG. 1, an internal view of the apparatus includes a looped conduit 15 is shown, situated within the apparatus to allow for the transmission of ultraviolet (UV) light in one direction and air and oxygen in the opposite direction. A UV lamp 2 is situated within the path of the conduit 15 to permit the light rays 3A, 3B and 3C, to reflect internally and uniformly by “total internal reflection” off the interior wall of conduit 15 so as to purify incoming air which is caused to move by a fan and motor 12. Total internal reflection may be accomplished by coating conduit 10 with a reflective coating, or by arranging an inner and an outer layer of two different refractive indices, so that total internal reflection occurs at the interfaces between the layers, as for example, in a fiber optic cable. Preferably, the air may undergo a first pass filtration by a filter system 14 equivalent to an N95 mask.

In the bottom portion of the apparatus, an electrolysis chamber may be provided to generate oxygen. The hydrogen molecule that is released from the reaction is directed to a second chamber where a chemical reaction can take place using calcium carbonate to generate Co₂ and calcium as a byproduct, thereby making the hydrogen gas innocuous. The water and CO₂ can be aerosolized from the system to reduce its weight:

CaCO₃(s)+2H³⁺(aq)→Ca²⁺(aq)+CO₂(g)+H₂O(l)

The hydrogen molecules may also be used to generate electricity using a hydrogen fuel cell, which is then used to operate the apparatus as described, continuously.

The apparatus is preferably fitted with a rechargeable or replaceable battery 11. A removable chamber is provided to add and remove calcium or any byproduct formed by a chemical reaction within the apparatus.

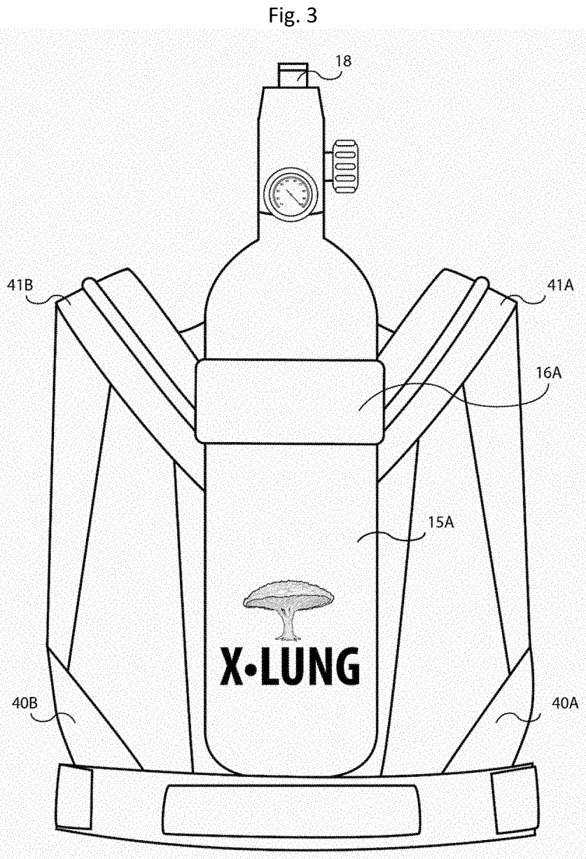

With further reference to FIG. 1, the mixed 100% sterilized air and oxygen is channeled out of the apparatus via port 18, as illustrated in FIG. 3.

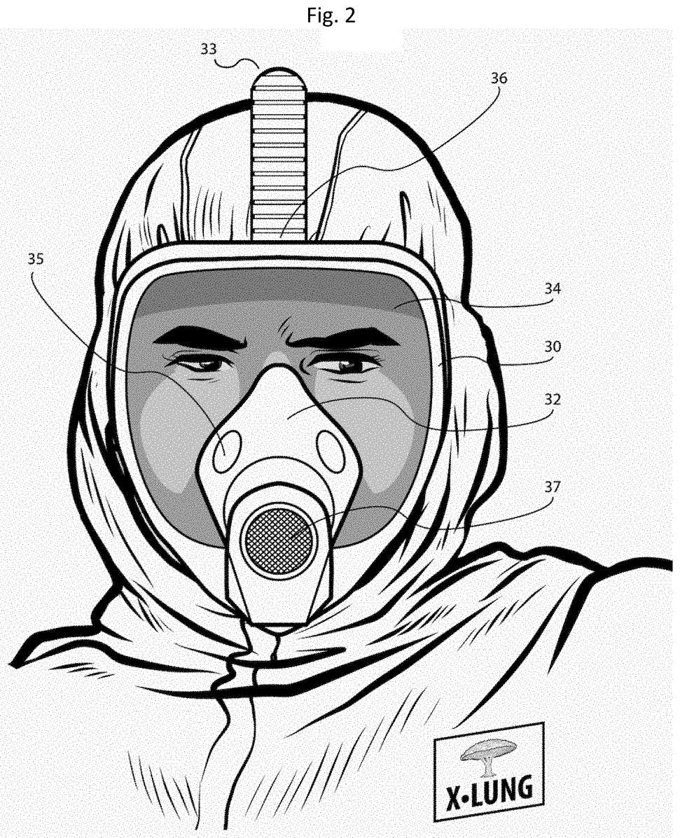

With reference to FIG. 2, a head covering face mask 30 is shown whereby the air and oxygen mixture will enter a conduit 33 which is attached to port 18 (FIG. 1) at one end and a port 36 at the other end. The air and oxygen mixture is thus channeled to the face via port 36. Visibility is provided by a transparent window 34. A one way valve may exist at 37, in a nose covering portion 32 affixed to and covering an opening in window 34, allows exhaled air to be removed from the face mask. The air, being high in CO₂, may be directed into a compartment within the apparatus to be used to facilitate a chemical reaction.

FIG. 3 is an external view of the apparatus depicting a harness including four body straps (two shoulder straps 41A and 41B, and two lower straps 40A and 40B, the latter being attached to a waist belt) which may permit the apparatus (shown as 15A, and secured to the harness by a circumferential or perimeter grip 16A in turn secured to the harness) to be worn on the back or the chest of a front line worker. A branch of the air conduit 18 may be directed towards a port to cool a full fitting body garment.

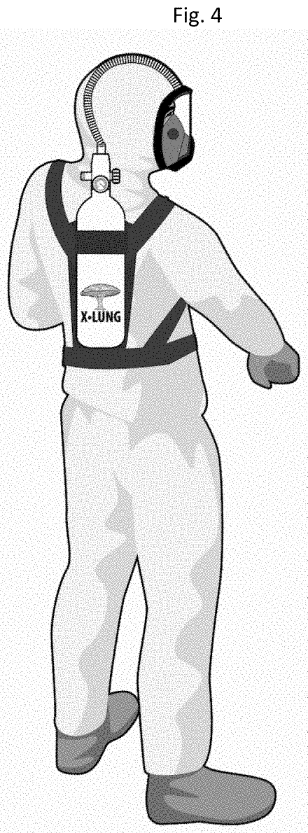

FIG. 4 is an illustration in which the apparatus of FIG. 1, connected to a shoulder harness of FIG. 3, can be worn in use.

The apparatus 15A can be miniaturized and disposed within the nose covering portion 32 (FIG. 2).

The technology as described can also be used for ventilators and personal nebulizer machines without departing from the principle of the invention.

An advantage of the apparatus disclosed herein is that there is a long path over which the air is exposed to UV light, thus permitting thorough elimination of pathogens before the user breathes the purified air. The longer path also increases the volume of the air that is purified. Another advantage of the apparatus described herein is that an N95 level filter may be used to pre-filter the air, which is then purified by being exposed to UV light.

I claim:

1. An air purification apparatus, comprising:
a source of ultraviolet radiation; and
a looping conduit having an internal surface exhibiting total internal reflection of the ultraviolet radiation for receiving the ultraviolet radiation from the source.

2. The air purification apparatus of claim 1, further comprising:
an N95 grade air filter barrier for pre-filtering air; and
an electric motor driven fan for moving the air through the filter barrier into the looping conduit.

3. The air purification apparatus of claim 1, further comprising a component within the apparatus for providing oxygen by electrolysis of water.

4. The air purification apparatus of claim 1, further comprising a hydrogen utilization apparatus to utilize the hydrogen to produce electricity.

5. The air purification apparatus of claim 4, wherein the hydrogen utilization apparatus is a fuel cell.

6. The air purification apparatus of claim 1, wherein air moves through the looping conduit in a first direction, and UV light moves in a second direction opposite to the first direction.

7. The air purification apparatus of claim 1, in combination with a harness for supporting the air purification apparatus.

8. The combination of claim 7, wherein the harness is worn by a person using the air purification apparatus.
